(19) United States
(12) Patent Application Publication
(10) Pub. No.: US 2026/0209192 A1
(43) Pub. Date: Jul. 23, 2026

(54) PROCESS FOR PREPARING AN N-SUBSTITUTED-2-OXAZOLIDINONE

(71) Applicant: HUNTSMAN PETROCHEMICAL LLC, The Woodlands, TX (US)

(72) Inventors: Hui ZHOU, The Woodlands, TX (US); Howard P. KLEIN, The Woodlands, TX (US)

(21) Appl. No.: 19/140,467

(22) PCT Filed: Dec. 13, 2023
(86) PCT No.: PCT/US2023/083746

§ 371 Date: Jun. 18, 2025

Related U.S. Application Data
(60) Provisional application No. 63/436,897, filed on Jan. 4, 2023.

(51) Int. Cl. C07D 263/22

(52) U.S. Cl. CPC C07D 263/22

(57) ABSTRACT

A process for preparing an N-substituted-2-oxazolidinone by reacting a cyclic carbonate and an N-substituted ethanolamine in the presence of an alkali carbonate catalyst to form the N-substituted-2-oxazolidinone.

### CROSS-REFERENCE TO RELATED APPLICATION

This application claims priority to U.S. Provisional No. 63/436,897 filed Jan. 4, 2023. The noted applications are incorporated herein by reference.

### FIELD

The present disclosure generally relates to a process for preparing N-substituted-2-oxazolidinones from cyclic carbonates and N-substituted ethanolamines and an alkali carbonate catalyst and their subsequent use in various applications, such as in lithium ion battery fabrication and as a solvent for electrode preparation.

### BACKGROUND

N-methyl-2-pyrrolidone (NMP) has been widely used in various industries, especially in the semiconductor, battery, and other electronics industries. NMP is a polar organic chemical with a unique combination of properties, such as low vapor pressure, comparatively high flash point, low freezing point and high boiling point. The molecular structure of NMP provides a particular combination of dispersive, polar, and hydrogen bonding forces that enable its unique solvency. However, the European Chemicals Agency (ECHA) has classified NMP as a “Substance of Very High Concern” because of its reproduction toxicity. Accordingly, there is a pressing need to replace NMP with lower toxicity alternatives having similar performance characteristics.

One potential alternative includes N-substituted-2-oxazolidinones. Various processes are known for producing such 2-oxazolidinone derivatives, for example by: reacting a β-aminoalcohol with one of phosgene, dialkyl carbonate, carbon dioxide, urea, isocyanate, ethylchlorocarbonate, or carbon disulfide; reacting an epoxide with cyanuric acid, urea or cyanamide; reacting an aziridine compound with carbon dioxide; or reacting acrolein with isocyanate.

However, such conventional processes have several disadvantages including a high cost of starting raw materials, the complexity of the procedure, the high toxicity of reactants and an overall low yield of the desired product. Therefore, there is a need for the development of a relatively simple, efficient process for the preparation of an N-substituted-2-oxazolidinone that does not suffer from such disadvantages.

### SUMMARY

The present disclosure generally provides a process for preparing an N-substituted-2-oxazolidinone. The process includes the step of reacting a cyclic carbonate and an N-substituted ethanolamine in the presence of an alkali carbonate catalyst to form a reaction product comprising the N-substituted-2-oxazolidinone. In some embodiments, the reaction product may be further subjected to a separation step to isolate the N-substituted-2-oxazolidinone from one or more by-products present in the reaction product.

The N-substituted-2-oxazolidinones produced according to the process of the present disclosure may be used in various applications, such as a solvent in battery, semiconductor and other electrochemical processing applications, or as an intermediate in the preparation of polymers, pharmaceuticals and agricultural chemicals.

### DETAILED DESCRIPTION

Before explaining aspects of the present disclosure in detail, it is to be understood that the present disclosure is not limited in its application to the details of construction and the arrangement of components or steps or methodologies set forth in the following description. The present disclosure is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those having ordinary skill in the art. Further, unless otherwise required by context, singular terms shall include pluralities and plural terms shall include the singular.

As utilized in accordance with the present disclosure, the following terms, unless otherwise indicated, shall be understood to have the following meanings.

The use of the word “a” or “an”, when used in conjunction with the term “comprising”, “including”, “having”, or “containing” (or variations of such terms) may mean “one”, but it is also consistent with the meaning of “one or more”, “at least one”, and “one or more than one”.

The use of the term “or” is used to mean “and/or” unless clearly indicated to refer solely to alternatives and only if the alternatives are mutually exclusive.

If the specification states a component or feature “may,” “can,” “could,” or “might” be included or have a characteristic, that particular component or feature is not required to be included or have the characteristic.

Throughout this disclosure, the term “about” is used to indicate that a value includes the inherent variation of error for the quantifying device, mechanism, or method, or the inherent variation that exists among the subject(s) to be measured. For example, but not by way of limitation, when the term “about” is used, the designated value to which it refers may vary by plus or minus ten percent, or nine percent, or eight percent, or seven percent, or six percent, or five percent, or four percent, or three percent, or two percent, or one percent, or one or more fractions therebetween.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrases “in one embodiment”, “in an embodiment”, “according to one embodiment”, and the like generally mean the particular feature, structure, or characteristic following the phrase is included in at least one embodiment of the present disclosure, and may be included in more than one embodiment of the present disclosure. Importantly, such phrases are non-limiting and do not necessarily refer to the same embodiment but, of course, can refer to one or more preceding and/or succeeding embodiments. For example, in the appended claims, any of the claimed embodiments can be used in any combination.

In the processes described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Values expressed in a range format should be interpreted in a flexible manner to include not only the numerical values explicitly recited as the limits of the range, but to also include all of the individual numerical values or sub-ranges encompassed within that range as if each numerical value and sub-range is explicitly recited. For example, a range such as from 1 to 6, should be considered to have specifically disclosed sub-ranges, such as, from 1 to 3, from 2 to 4, from 3 to 6, etc., as well as individual numbers within that range, for example, 1, 2, 3, 4, 5, and 6. This applies regardless of the breadth of the range.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the present disclosure.

The term “optional” or “optionally” means that the subsequently described event or circumstance may or may not occur, and that the description includes instances where said event or circumstance occurs and instances where it does not.

The present disclosure is generally directed to a process for the preparation of an N-substituted-2-oxazolidinone including the step of reacting a cyclic carbonate with an N-substituted ethanolamine in the presence of an alkaline metal catalyst to form a reaction product comprising the N-substituted-2-oxazolidinone.

According to one embodiment, the cyclic carbonate is a compound having the formula (I):

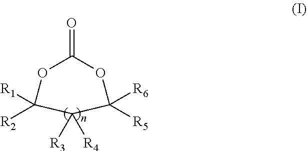

where R₁, R₂, R₃, R₄, R₅ and R₆ are independently selected from hydrogen, a hydroxyalkyl group, and a hydrocarbyl group having from 1 to 8 carbon atoms; and n is an integer from zero to one. In one embodiment, R₁, R₂, R₃, R₄, R₅ and R₆ are independently selected from hydrogen and a hydrocarbyl group having from 1 to 4 carbon atoms, and more preferably are independently selected from hydrogen, a methyl group, an ethyl group and a propyl group.

In other embodiments, the cyclic carbonates for use in this disclosure are those of formula I above where n is zero and where R₁, R₂, R₃, R₄ and R₆ are hydrogen and R₅ is hydrogen, methyl, ethyl or hydroxymethyl. In further embodiments when n is 1, R₁, R₂, R₃, R₄, R₅ and R₆ are independently hydrogen, methyl or ethyl. Most preferred cyclic carbonates are ethylene carbonate, propylene carbonate and the butylene carbonate which are defined below.

The following are examples of cyclic carbonates for use in this disclosure and including mixtures thereof: 1,3-dioxolan-2-one (also referred to as ethylene carbonate); 4-methyl-1,3-dioxolan-2-one (also referred to as propylene carbonate); 4-hydroxymethyl-1,3-dioxolan-2-one; 4,5-dimethyl-1,3-dioxolan-2-one; 4-ethyl-1,3-dioxolan-2-one; 4,4-dimethyl-1,3-dioxolan-2-one (previous three also referred to as butylene carbonates); 4-methyl-5-ethyl-1,3-dioxolan-2-one; 4,5-diethyl-1,3-dioxolan-2-one; 4,4-diethyl-1,3-dioxolan-2-one; 1,3-dioxan-2-one; 4,4-dimethyl-1,3-dioxan-2-one; 5,5-dimethyl-1,3-dioxan-2-one; 5,5-dihydroxymethyl-1,3-dioxan-2-one; 5-methyl-1,3-dioxan-2-one; 4-methyl-1,3-dioxan-2-one; 5-hydroxy-1,3-dioxan-2-one; 5-hydroxymethyl-5-methyl-1,3-dioxan-2-one; 5,5-diethyl-1,3-dioxan-2-one; 5-methyl-5-propyl-1,3-dioxan-2-one; 4,6-dimethyl-1,3-dioxan-2-one; and 4,4,6-trimethyl-1,3-dioxan-2-one.

In one embodiment, the N-substituted ethanolamine which is reacted with the cyclic carbonate is a compound having the formula (II):

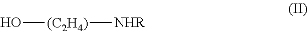

where R is a hydrocarbyl group having from 1 to 10 carbon atoms, a cycloalkyl group, an aralkyl group or a hydroxyalkyl group. In some embodiments, R is a hydrocarbyl group having from 1 to 6 carbon atoms, such as a methyl group, an ethyl group, a propyl group, an isopropyl group or a butyl group. In other embodiments, R is a cycloalkyl group, such as a cyclohexyl group or a methylcyclohexyl group. In still other embodiments, R is an aralkyl group, such as a benzyl group. In still further embodiments, R is a hydroxyalkyl group, such as a hydroxymethyl group, a hydroxyethyl group or a hydroxypropyl group.

In one particular embodiment, the N-substituted ethanolamine is selected from 2-(methylamino) ethanol, 2-(ethylamino) ethanol, 2-(butylamino) ethanol, 2-(benzylamino) ethanol and 2-(cyclohexylamino)ethanol.

The reaction between the cyclic carbonate and N-substituted ethanolamine takes place in the presence of an alkali carbonate catalyst. Examples of alkali carbonate catalysts include potassium carbonate, sodium carbonate, rubidium carbonate, cesium carbonate, beryllium carbonate, magnesium carbonate, calcium carbonate, strontium carbonate, manganese carbonate, barium carbonate, sodium hydrogencarbonate, potassium hydrogencarbonate, lithium hydrogencarbonate, calcium hydrogencarbonate, barium hydrogencarbonate, magnesium hydrogencarbonate, strontium hydrogencarbonate and combinations thereof. In one embodiment, the alkali carbonate catalyst is selected from potassium carbonate, sodium carbonate, calcium carbonate and potassium hydrogencarbonate, and in one preferred embodiment is potassium carbonate.

The reaction between the cyclic carbonate and N-substituted ethanolamine may take place in the absence or presence of a solvent which does not participate in the reaction. Preferably a solvent is not used.

In some embodiments, the molar ratio of the cyclic carbonate to the N-substituted ethanolamine present during the reaction may be from about 0.8:1 to about 1:1.2, or from about 0.9:1 to about 1.1:1. In other embodiments, the molar ratio of the cyclic carbonate to the N-substituted ethanolamine present during the reaction may be from about 0.95:1 to about 1.05:1, or from about 0.97:1 to about 1.03:1, or from about 0.99:1 to about 1.01:1. The order of addition of the cyclic carbonate and the N-substituted ethanolamine as raw materials is not particularly limited, and the cyclic carbonate may be added to the N-substituted ethanolamine, or the N-substituted ethanolamine may be added to the cyclic carbonate or the N-substituted ethanolamine and the cyclic carbonate may be added at once.

In one embodiment, the amount of the alkali carbonate catalyst present during the reaction may be less than about 2% by weight, or less than about 1.5% by weight, or less than about 1% by weight, or less than about 0.5% by weight, based on the total weight of the cyclic carbonate, the N-substituted ethanolamine and the alkali carbonate catalyst (i.e., “total weight of the reaction mixture”). In other embodiments, the amount of the alkali carbonate catalyst present during the reaction may be from about 0.001% by weight to about 1% by weight, or from about 0.01% by weight to about 0.75% by weight, or from about 0.02% by weight to about 0.1% by weight, based on the total weight of the reaction mixture.

In some embodiments, the reaction between the cyclic carbonate and N-substituted ethanolamine may take place at a temperature of from about 40° C. to about 150° C., or from about 50° C. to about 130° C. The reaction between the cyclic carbonate and the N-substituted ethanolamine may take place under pressure, or at reduced pressure, but is preferably carried out at atmospheric pressure. The time for the reaction to reach completion may be from about 0.5 hours to about 10 hours, or from about 2 hours to about 5 hours. In addition to the desired N-substituted-2-oxazolidinone, one or more by-products may be present in the obtained reaction product (for e.g., one or more of by-product dihydroxyamine, unreacted N-substituted ethanolamine, unreacted cyclic carbonate, by-product dialkylene glycol, by-product trialkylene glycol and other impurities). The N-substituted-2-oxazolidinone can be isolated from at least a portion of the one or more by-products and purified by subjecting the reaction product to a separation technique, such as a concentration, crystallization, recrystallization, distillation, fractional distillation, or a chromatography technique.

The percent yield of the N-substituted-2-oxazolidinone (i.e., 100×([measured amount of N-substituted-2-oxazolidinone]/[maximum amount of N-substituted-2-oxazolidinone that can be produced from the given amounts of cyclic carbonate and N-substituted ethanolamine])) obtained by the process of the present disclosure may be at least about 85%, or at least about 90%, or at least about 92%, or at least about 93%, or at least about 94% or at least about 95%. Particular examples of N-substituted-2-oxazolidinones which may be produced according to the process of the present disclosure include, but are not limited to, N-methyl-2-oxazolidinone, N-ethyl-2-oxazolidinone, N-propyl-2-oxazolidinone, N-isopropyl-2-oxazolidinone, N-butyl-2-oxazolidinone, N-hydroxyethyl-2-oxazolidinone, N-hydroxypropyl-2-oxazolidinone N-cyclohexyl-2-oxazolidinone and N-benzyl-2-oxazolidinone.

According to one embodiment, approximately equimolar (i.e., 0.99:1 to 1.01:1) amounts of a cyclic carbonate and N-substituted ethanolamine are combined and allowed to initially react at a temperature of from about 50°-70° C. and atmospheric pressure for a period of time of less than about 1 hour. An alkali carbonate catalyst is then added and the cyclic carbonate and N-substituted ethanolamine are further reacted at a temperature of from about 120°-140° C. and at atmospheric pressure until completion of the reaction (which may be confirmed by gas chromatography). At least a portion of the one or more by-products present in the obtained reaction product are then removed by distillation to produce the N-substituted-2-oxazolidinone having a purity of at least about 90%, or at least about 95% or at least about 99%.

The N-substituted-2-oxazolidinones obtained by the process of the present disclosure may be used in a variety of ways, such as in the preparation of a lithium ion battery, as a photochemical reaction solvent, as a solvent for photoelectrochemical display elements, in an electrolyte solvent, in an electrolyte solvent for batteries, as a solvent for electrolytic reactions, as a solvent for electrolytic polymerization, as a solvent for electroplating, as a solvent for electrolytic polishing, as an aprotic polar solvent in organic synthesis reactions, as a solvent for polymerization or extraction, as a low toxic high-boiling solvent, as an industrial cleaning agent, as a solvent for peeling a coating film, as a pigment dispersant, and as an intermediate in the preparation of polymer materials, pharmaceuticals and agricultural chemicals. For example, the N-substituted-2-oxazolidinones are useful as intermediates in the production of fibers, tablet coatings, lubricant additives, rust inhibitors and dyeing assistants.

Examples of the present process for preparing N-substituted-2-oxazolidinones are provided below. However, the present disclosure is to be understood to not be limited in its application to the specific experiments, results, and laboratory procedures disclosed herein below. Rather, the Examples are simply provided as one of various embodiments and are meant to be exemplary and not exhaustive.

### EXAMPLES

### Example 1—Process for Preparing N-methyl-2-oxazolidinone

A 1 liter 3-neck round bottom flask with an agitator, addition funnel, nitrogen inlet, condenser, and 1 inch ID distillation column, was charged with 400 grams propylene carbonate. The propylene carbonate was then heated to 50°-70° C. 294 grams of 2-(methylamino) ethanol was then slowly added to the flask via the addition funnel and the reaction temperature was maintained within a range of 50°-70° C. for about 0.5 hours. 0.14 grams of potassium carbonate was then charged to the flask and the reaction temperature was slowly adjusted to and maintained at 130° C. for about 3 hours The crude reaction product was distilled to 99% pure N-methyl-2-oxazolidinone and co-product, propylene glycol. The % yield of 3-methyl-2-oxazolidinone was high (>93%), based on gas chromatography measurements. Example 2—Process for preparing N-ethyl-2-oxazolidinone

A 1 liter 3-neck round bottom flask with an agitator, addition funnel, nitrogen inlet, condenser, and 1 inch ID distillation column, was charged with 1030 grams propylene carbonate. The propylene carbonate was then heated to 50°-70° C. 899 grams of 2-(ethylamino) ethanol was then slowly added to the flask via the addition funnel and the reaction temperature was maintained within a range of 50°-70° C. for about 0.5 hours. 0.58 grams of potassium carbonate was then charged to the flask and the reaction temperature was slowly adjusted to and maintained at 130° C. for about 3 hours The crude reaction product was distilled to 99% pure 3-ethyl-2-oxazolidinoneand and coproduct, propylene glycol. The % yield of 3-ethyl-2-oxazolidinone was high (>93%), based on gas chromatography measurement.

### Example 3—Process for Preparing N-butyl-2-oxazolidinone

A 1 liter 3-neck round bottom flask with an agitator, addition funnel, nitrogen inlet, condenser, and 1 inch ID distillation column, was charged with 1481 grams propylene carbonate. The propylene carbonate was then heated to 50°−70° C. 1700 grams of 2-(butylamino) ethanol was then slowly added to the flask via the addition funnel and the reaction temperature was maintained within a range of 50°-70° C. for about 0.5 hours. 0.95 grams of potassium carbonate was then charged to the flask and the reaction temperature was slowly adjusted to and maintained at 130° C. for about 3 hours The crude reaction product was distilled to 99% pure 3-butyl-2-oxazolidinoneand and coproduct, propylene glycol. The % yield of 3-butyl-2-oxazolidinone was high (>95%), based on gas chromatography measurement

From the above description, it is clear that the present disclosure is well adapted to carry out the object and to attain the advantages mentioned herein as well as those inherent in the present disclosure. While exemplary embodiments of the present disclosure have been described for the purposes of the disclosure, it will be understood that numerous changes may be made which will readily suggest themselves to those skilled in the art which can be accomplished without departing from the scope of the present disclosure and the appended claims.

What is claimed is:

1. A process for preparing an N-substituted-2-oxazolidinone comprising reacting a cyclic carbonate with an N-substituted ethanolamine in the presence of an alkali carbonate catalyst to form a reaction product comprising the N-substituted-2-oxazolidinone.

2. The process according to claim 1, wherein the cyclic carbonate is a compound having the formula (I): 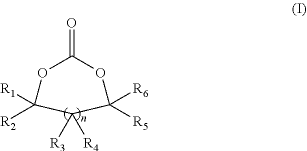
wherein R₁, R₂, R₃, R₄, R₅ and R₆ are independently selected from hydrogen, a hydroxyalkyl group, and a hydrocarbyl group having from 1 to 8 carbon atoms; and n is an integer from zero to one.

3. The process according to claim 2, wherein n is zero.

4. The process according to claim 3, wherein R₁, R₂, R₃, R₄, R₅ and R₆ are independently selected from hydrogen and a hydrocarbyl group having from 1 to 4 carbon atoms.

5. The process according to claim 4, wherein R₁, R₂, R₃, R₄, R₅ and R₆ are independently selected from hydrogen, a methyl group, an ethyl group and a propyl group.

6. The process according to claim 1, wherein the N-substituted ethanolamine is a compound having the formula (II): 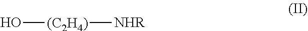
wherein R is a hydrocarbyl group having from 1 to 10 carbon atoms, a cycloalkyl group, an aralkyl group or a hydroxyalkyl group.

7. The process according to claim 6, wherein R is a hydrocarbyl group having from 1 to 6 carbon atoms.

8. The process according to claim 7, wherein R is a methyl group, an ethyl group, a propyl group, an isopropyl group or a butyl group.

9. The process according to claim 1, wherein the alkali carbonate catalyst is selected from potassium carbonate, sodium carbonate, calcium carbonate and potassium hydrogencarbonate.

10. The process according to claim 9, wherein the alkali carbonate catalyst is potassium carbonate.

11. A process for preparing an N-substituted-2-oxazolidinone comprising reacting a cyclic carbonate with an N-substituted ethanolamine in the presence of an alkali carbonate catalyst to form a reaction product comprising the N-substituted-2-oxazolidinone and one or more by-products and subjecting the reaction product to a separation technique to remove at least a portion of the one or more by-products.

12. The process according to claim 11, wherein the cyclic carbonate and the N-substituted ethanolamine are present during the reaction at a molar ratio of from about 0.99:1 to about 1.01:1.

13. The process according to claim 11, wherein the amount of the alkali carbonate catalyst present during the reaction is from about 0.005% by weight to about 1% by weight, based on the total weight of the cyclic carbonate, N-substituted ethanolamine and alkali carbonate catalyst.

14. The process according to claim 11, wherein the reaction takes place at a temperature of from about 50° C. to about 130° C.

15. The process according to claim 11, wherein the separation technique is a concentration, crystallization, recrystallization, distillation, fractional distillation, or chromatography technique.

16. The process according to claim 11, wherein the N-substituted-2-oxazolidinine is N-methyl-2-oxazolidinone, N-ethyl-2-oxazolidinone, N-propyl-2-oxazolidinone, N-isopropyl-2-oxazolidinone, N-butyl-2-oxazolidinone, N-hydroxyethyl-2-oxazolidinone, N-hydroxypropyl-2-oxazolidinone N-cyclohexyl-2-oxazolidinone or N-benzyl-2-oxazolidinone.

17. The process according to claim 16, wherein the N-substituted-2-oxazolidinone is prepared at a percent yield of at least 85%.
